(19) United States
(12) Patent Application Publication
(10) Pub. No.: US 2003/0033639 A1
(43) Pub. Date: Feb. 13, 2003

(54) Method for introducing gene into plant having improved transformation efficiency

(75) Inventors: Etsuko Matsunaga, Tokyo (JP); Koichi Sugita, Tokyo (JP); Hiroyasu Ebinuma, Tokyo (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: NIPPON PAPER INDUSTRIES CO., LTD., Kita-ku (JP)

(21) Appl. No.: 10/201,110

(22) Filed: Jul. 24, 2002

(30) Foreign Application Priority Data
Jul. 24, 2001 (JP) 2001-223664

(51) Int. Cl. A01H 1/00

(52) U.S. Cl. 800/294

(57) ABSTRACT

A method for producing a transgenic plant, which comprises: preparing a shoot having at least one end having a growing point and a base part having no growing point as a tissue for gene introduction; and introducing a desired gene into the base part while keeping the growing point in the at least one end to differentiate an adventitious bud.

### BACKGROUND OF THE INVENTION

1. Field of the Invention

The present invention relates to a method for improving transformation efficiency in the introduction of a desired gene into a plant using genetic engineering techniques.

2. Description of the Background

According to gene introduction into a plant using genetic engineering techniques, a desired gene can be directly introduced into a plant. Accordingly, it has many advantages over the classical breeding which repeats crossing. For example, according to the gene introduction, (a) only a character to be modified can be introduced, (b) characters of species other than plants (microorganisms and the like) can also be introduced into a plant, and (c) a breeding period can be markedly reduced.

However, there are many plants in that the advantages cannot be obtained sufficiently depending on the kind of the tree due to low transformation efficiency by genetic engineering techniques. For example, since trees have a long life cycle, the conventional bleeding which is carried out by repeating crossing requires a vast land and a long years. Also, trees are one of the most important biomass resources and expected in recent years to contribute to the maintenance and improvement of terrestrial environment, so that concern has been directed toward the development of varieties having more excellent properties such as productivity, environmental resistance and the like. Thus, although the bleeding by gene introduction produces large advantages also in trees, the transformation efficiency into industrially important tree species is very low in most cases.

Accordingly, for transformation of such trees, attempts have been made to improve the transformation efficiency through examinations from various points of view, such as kinds of tissues to be used, conditions for the gene introduction treatment and culture conditions of the tissue before and after the treatment. A method in which a gene introduction treatment is carried out using a shoot as the tissue for gene introduction, and an adventitious bud is re-differentiated therefrom to obtain a desired gene-introduced individual, namely a transformant, has also been examined on tree species.

However, it is also difficult to improve the efficiency of tree species which have originally low transformation efficiency. After all, actually, an industrially important tree species whose properties have been improved to practically valuable levels by genetic engineering techniques has not been obtained yet.

Also, up to date, the methods already established as the preparation method of transformed plants are those which had been established by defining the gene introduction treatment conditions, culture conditions of tissues for gene introduction and the like, in response to the respective kinds of plants to be used, and attempting to improve the transformation efficiency individually. A transformation method which can improve the transformation efficiency common to a number of plants is also not known yet.

### SUMMARY OF THE INVENTION

An object to the present invention is to provide a method for producing transgenic plants applied to a number of plants including trees in which transformation is considered to be difficult, by improving transformation efficiency using genetic engineering techniques.

This and other objects of the present invention have been accomplished by a method for producing a transgenic plant, which comprises:

preparing a shoot having at least one end having a growing point and a base part having no growing point as a tissue for gene introduction; and

introducing a desired gene into the base part while keeping the growing point in the at least one end to differentiate an adventitious bud.

### BRIEF DESCRIPTION OF THE DRAWINGS

FIG. 1 is a schematic view showing the structure of a T-DNA region which is incorporated into a plant gene in pBI121 vector.

FIG. 2 is a schematic view showing the structure of a T-DNA region which is incorporated into a plant gene in pIPT10 vector.

### DETAILED DESCRIPTION OF THE INVENTION

As a result of intensive studies, the present inventors have found that when a shoot having a growing point is used as a tissue for gene introduction and a desired gene is introduced into a base part of the shoot, the desired gene is efficiently introduced into a cell of the base part and furthermore, the base part of the shoot to which the desired gene has been thus introduced has high ability to redifferentiate a desired gene-introduced adventitious bud. Thus, the present invention has been accomplished.

The present invention can be applied to any plants regardless of their species. However, effects of the present invention can be particularly obtained in plants in which gene introduction is considered to be difficult, such as trees and the like.

In the present invention, the shoot to be used as the tissue for gene introduction means an elongated normal bud or adventitious bud. Also, in order to further improve the transformation efficiency, it is preferable to use, as the tissue for gene introduction, a tissue or part having high redifferentiation potency such as a young tissue having a low content of polyphenols which become the cause of browning of tissues. From this point of view, a hypocotyl obtained by germinating a seed and an elongated apical bud or lateral bud of a plant cultured in a culture vessel are selected in the present invention as suitable tissues for gene introduction.

However, the shoot must have at least one end having a growing point and a base part having no growing point. For example, when a hypocotyl is used as the tissue for gene introduction of the present invention, the shoot can be obtained by cutting off a root and a part having a base of root from a hypocotyl after germination of a seed while leaving the apical bud and apical bud primordium as they are. In this case, the one end having a growing point means a shoot end having an apical bud and the like, and the base part having no growing point means a part having the end face formed by cutting off a root and the like. Also, when an elongated apical bud or lateral bud of a plant is used as the tissue for gene introduction of the present invention, it can be obtained by simply cutting out from the plant. In this case, the one end having a growing point means a shoot end having an apical bud and the like similar to the case of the hypocotyl, and the base part having no growing point means a part having the face cut out from the plant. That is, the desired gene is preferably introduced into the end face of the base part of the shoot generated when a shoot is prepared as the tissue for gene introduction.

In the present invention, an adventitious bud is differentiated by introducing a desired gene into the base part having no growing point, while keeping the growing point of at least one end. As the desired gene, various genes such as a gene which can provide an industrially excellent character and a gene which cannot always provide an industrially excellent character but is necessary in studying gene expression mechanism can be selected and used.

A desired gene can be introduced into the base part of the shoot indirectly via Gemini virus, Brome mosaic virus, Agrobacterium tumefaciens (hereinafter referred to as “A. tumefaciens”), Agrobacterium rhizogenes and the like viruses and bacteria, by inserting the desired gene into an appropriate vector, or directly by the particle gun method and the like (I. Potrykus, Annu. Rev. Plant Physiol. Plant Mol. Biol., 42: 205 (1991)).

For example, in a gene introduction method using Agrobacterium (Agrobacterium method, e.g., EP-A-0290799, U.S. Pat. Nos. 4,940,838 and 5,591,616 and the like), an appropriate vector into which a desired gene has been inserted is introduced into Agrobacterium in advance, and the desired gene is introduced into a tissue for gene introduction by infection with the Agrobacterium. The infection of the Agrobacterium is carried out, for example, by soaking the tissue for gene introduction in a solution in which the Agrobacterium is suspended.

Also, since the infection of the Agrobacterium occurs in a wound of the plant tissue, when a shoot in which a wound is formed on its base part is soaked in an Agrobacterium suspension, the wound is infected with the Agrobacterium and the desired gene is introduced into the base part of the shoot. When a hypocotyl is used as the tissue for gene introduction by cutting off roots and the like or elongated apical bud or the like is used by cutting out it from a plant, the infection occurs at the end face (cut surface) of the base part of the shoot formed by the cutting. That is, in these cases, the desired gene is introduced into the base part of the shoot by merely soaking the tissue for gene introduction simply in the Agrobacterium suspension.

Furthermore, a plant tissue is surely infected with the Agrobacterium when a tissue for gene introduction is soaked in the cell suspension and then cocultured with the Agrobacterium for several days by introducing the tissue on a solid medium. As the coculturing medium, a well known basal medium such as MS (Murashige and Skoog, Physiol. Plant, 15: 473-497 (1962)) or WPM (Loyd and McCown, Prop. Int. Plant Prop. Soc., 30:421-427 (1980)), or a modified composition thereof to suit for the tissue for gene introduction to be infected with the Agrobacterium, can be used by supplementing it with a carbon source and a medium solidifying agent and, if necessary, with plant hormones such as auxins, cytokinins and the like appropriately. In this case, generally, 10 to 30 g/l sucrose is used as the carbon source; 5 to 10 g/l agar or 1 to 4 g/l gelan gum is used as the medium solidifying agent; 0.01 to 5.0 mg/l zeatin, benzyladenine or the like is used as the cytokinins; and 0.01 to 2.0 mg/l naphthaleneacetic acid (NAA), indolebutyric acid (IBA), indoleacetic acid or the like is used as the auxins. Also, infectivity of the Agrobacterium is increased in some cases by adding 10 to 200 mg/l acetosyringon to the above coculturing medium.

On the other hand, when a desired gene is introduced by a particle gun, the above coculturing medium can be used as the medium for gene introduction treatment. In this case, a tissue for gene introduction is placed on the medium with its position into which the desired gene is to be introduced, namely the base part of the shoot, upside, and the gene introduction is carried out by manipulating the particle gun in the usual way.

In general, when a desired gene is introduced into a tissue for gene introduction, a selectable marker gene is introduced together with the desired gene, and expression of the selectable marker gene is used as an index of the introduction of the desired gene. In the method of the present invention, the transformation efficiency can be further improved by using a cytokinin-related gene as the selectable marker gene.

Herein, the cytokinin-related gene is a gene which acts in a direction of increasing the influence of cytokinin in the introduced plant cells and thereby increases adventitious bud differentiation ability of the cells. Examples of the gene include the ipt gene as an A. tumefaciens-derived cytokinin synthesis gene (A. C. Smigocki and L. D. Owens, Proc. Natl. Acad. Sci. USA, 85:5131 (1988)), the Escherichia coli-derived β-glucuronidase gene as a gene which activates inactive cytokinin (Morten Loersbo and Finn T. Okkels, Plant Cell Reports, 16: 219-221 (1996)), the Arabidopsis thaliana-derived CK11 gene which is considered to be a cytokinin receptor gene (Kakimoto T., Science, 274: 982-985 (1996)) and the like. Particularly, the ipt gene used in Examples of the present specification is a gene which is most well known and whose function has been revealed.

Also, the desired gene, the cytokinin-related gene and other nucleotide sequences and genes which are optionally introduced may be introduced by inserting them into the same vector or be introduced by inserting them into different vectors with no problems, so long as they are incorporated into the same cells of the tissue for gene introduction. However, when the genes and the like are incorporated into the same vector, it is necessary to arrange them such that the presence of one side of genes and the like does not inhibit expression of the other side of genes and the like.

An adventitious bud can be differentiated from a tissue after the gene introduction by culturing the tissue using an appropriate medium. A composition of the medium suitable for the adventitious bud differentiation varies depending on each plant, but in the case of the genus Eucalyptus, the MS medium in which the concentration ratio of ammonia nitrogen and nitrate nitrogen is changed to 1:3 (hereinafter simply referred to as “modified MS medium”) can be used as the medium for adventitious bud differentiation (the shoot regeneration medium) after diluting it to 1 to 4 folds and supplementing it with 10 to 30 g/l sucrose, 1 to 4 g/l gelan gum or 5 to 10 g/l agar, and 0.2 to 5.0 mg/l zeatin and 0.01 to 1.0 mg/l NAA as plant hormones. However, when a cytokinin-related gene is used as the selectable marker gene, the plant hormones may not be added (may be hormone-free) or auxin alone may be added. Also, when a gene is introduced by the above Agrobacterium method, antibiotics such as carbenicillin, ticarcillin, cefotaxime and the like are added to the medium in an amount of 10 to 10,000 mg/l to inhibit the Agrobacterium growth. It is preferable that the temperature is from 15 to 30° C. and the light intensity is from 0 to 200 μmol/m²/s. Since the growing point of a shoot preserved at the time of the gene introduction is not particularly required in the subsequent steps, it can be cut off at an appropriate stage.

The tissue cultured using the shoot regeneration medium differentiation differentiates the adventitious bud generally several weeks after commencement of the culturing. In this case, a callus may grow slightly prior to the adventitious bud differentiation. The desired gene is introduced into the thus differentiated adventitious bud at higher frequency than the differentiated adventitious bud differentiated by introducing the gene into a segment based on the conventional method. However, when the gene introduction is carried out using a cytokinin-related gene as a selectable marker gene, the adventitious bud into which the desired gene has been introduced may sometimes show morphological abnormality such as multiple bud or the like due to the induction of morphological abnormality by the gene. Even in that case, however, an adventitious bud having normal morphology can finally be obtained by removing influence of the cytokinin-related gene when the cytokinin-related gene is used in combination with a DNA factor having leaving ability as shown, for example, in JP-A-9-154580.

A plantlet into which the desired gene has been introduced can be regenerated by cutting out the thus obtained adventitious bud and transplanting it on a rooting medium containing, for example, 0 to 1.0 mg/ml auxins, for rooting.

The present invention is based on the knowledge that a desired gene is efficiently introduced into cells of a base part of a shoot having no growing point when the base part is subjected to a gene introduction treatment while keeping a growing point on at least one end of the shoot, and that the base part of the shoot into which the desired gene has been introduced in this manner has higher ability to redifferentiate an adventitious bud into which the desired gene has been introduced, in comparison with the case of introducing the desired gene into the base of the shoot from which growing point has been removed.

The reason for this is not necessarily clear. However, it is considered that the growing point which is present in at least one end of the same shoot is contributing to this in some forms. Since plant tissues and cells are always damaged at a certain degree in carrying out gene introduction and the recovering strength from this damage is reinforced by the presence of a growing point, it seems that active growth ability is also maintained in the gene-introduced cells, and thereby acts advantageously on the introduction of the desired gene, and/or redifferentiation of an adventitious bud into which the desired gene has been introduced.

In addition, the base part of the shoot having no growing point is generally considered to be a part suitable for rooting. Accordingly, it is considered that when a gene is introduced into the part using a cytokinin-related gene as the selectable marker gene, difference in the ability differentiating an adventitious bud becomes sharply large between the gene-introduced cells and not-introduced cells and, as a result, the cells into which the desired gene has been introduced selectively differentiate adventitious buds. Thus, the use of such a gene as the selectable marker gene more advantageously results in the differentiation of the adventitious bud into which the desired gene has been introduced.

According to the present invention, introduction efficiency of a desired gene can be improved by the method for introducing a gene into a plant. Furthermore, in the present invention, an adventitious bud into which a desired gene has been introduced are differentiated efficiently from a tissue introduced with the desired gene. Thus, according to the present invention, introduction efficiency of the desired gene into an adventitious bud is particularly improved.

The present invention can be applied to many plants, and the effect of the present invention has a great meaning particularly for a plant in which gene introduction has been considered to be difficult.

That is, according to the present invention, gene introduction into industrially important tree species can be carried out and the present invention opens a way for preparing a transformant which can be practically valued.

The present invention is explained below based on Examples in details; however, the present invention is not limited thereto.

### EXAMPLE 1

Seeds of Eucalyptus camaldulensis (hereinafter referred to as “E. camaldulensis”) were sterilized by soaking them in 70% ethanol for 1 minute and further soaking in 2% aqueous sodium hypochlorite solution for about 2 hours with stirring, washed thoroughly with sterile water and inoculated onto a germination medium, preserved for 2 days or more in a refrigerator of 4° C. for accelerating germination, and then cultured at 25° C. under whole light condition of 40 μmol/m²/s in light intensity to effect their germination. In this case, a 2-fold diluted modified MS medium (hereinafter referred to as “camaldulensis basal medium”) was supplemented with 10 g/l sucrose and 8 g/l agar and used as the germination medium.

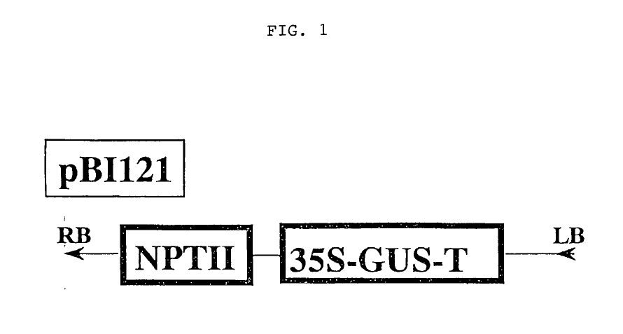

One to two weeks after the inoculation of seeds onto the germination medium, roots and seed leaves were cut off from the thus germinated seedlings to collect each hypocotyl keeping back apical bud alone on its one end, and a pBI121 vector (available from CLONTECH, cf. FIG. 1) was introduced into its base part having no growing point. In FIG. 1, NPTII indicates a kanamycin-resistant gene, and 35S-GUS-T indicates a GUS gene to which a 35S promoter and a terminator are connected at the 5′ side and at the 3′ side, respectively. That is, the pBI121 vector was introduced into A. tumefaciens EHA105 in advance by electroporation (using GENE PULSER II manufactured by Bio-Rad), followed by culturing overnight in YEB liquid medium and diluted to OD₆₃₀=0.5 with the camaldulensis basal medium to prepare a cell suspension, and then the hypocotyl collected in the above manner was soaked in the cell suspension. Next, after discarding excess cell suspension, the hypocotyl was cocultured with the Agrobacterium at 25° C. for 2 days in the dark using the shoot regeneration medium supplemented with 40 mg/l acetosyringon, to thereby infect it with the pBI121 vector-introduced Agrobacterium. In this case, the camaldulensis basal medium was supplemented with 2.0 mg/l zeatin, 0.3 mg/l NAA, 10 g/l sucrose and 8 g/l agar and used as the shoot regeneration medium.

The pBI121 vector used herein is a vector prepared by inserting the GUS gene as a model of the desired gene and a kanamycin-resistant gene (NPTII gene) as the selectable marker gene. Thus, the cells are introduced with the GUS gene and kanamycin-resistant gene by the introduction of the pBI121 vector, and show GUS activity and kanamycin resistance.

The hypocotyl after coculturing was transplanted onto the shoot regeneration medium further supplemented with 500 mg/l ticarcillin and 50 mg/l kanamycin for the selection of pBI121 vector-introduced cells and cultured at 25° C. under whole light conditions of 40 μmol/m²/s in light intensity by sub-culturing it using the same medium composition at an interval of 2 weeks, and 3 months after the Agrobacterium infection, the thus differentiated and elongated adventitious bud was rooted using a rooting medium. As the rooting medium, the Eucalyptus basal medium was used after supplementing it with 0.05 mg/l IBA, 500 mg/l ticarcillin, 150 mg/l kanamycin, 10 g/l sucrose and 2.5 g/l gelan gum.

When the experimentation was repeated three times on 50 hypocotyls, differentiation of adventitious buds was started to be observed 6 weeks after the Agrobacterium infection in each case, and finally, rooting from buds originated from 6 of the 150 hypocotyls (hereinafter referred to as “buds of 6 lines”) was observed after 4 months. When a GUS activity test was carried out on the buds in accordance with the method of Jefferson et al., its expression was confirmed in buds of 5 lines. That is, the transformation efficiency in this case was 6/150×100=4.0% based on the kanamycin resistance and 5/150×100=3.3% based on the GUS activity. The results are shown in Table 1.

### COMPARATIVE EXAMPLE 1

Introduction of the GUS gene and kanamycin-resistant gene and subsequent regeneration of plants were carried out in the same manner as in Example 1, except that those which were prepared by cutting off roots, cotyledons and apical buds from the germinated seedlings of E. camaldulensis seeds were used as the hypocotyls for gene introduction.

As a result of the test on 320 hypocotyls, rooting was observed finally in buds of 8 lines, and the GUS activity was observed in buds of 6 lines among them. That is, the transformation efficiency in this case was 8/320×100=2.5% based on the kanamycin resistance and 6/320×100=1.9% based on the GUS activity. The results are shown in Table 1.

TABLE 1

| | Ex. 1 | Comp. Ex. 1 |
|---|---|---|
| Plant | E. camaldulensis | |
| Desired gene | GUS gene | |
| Selectable marker gene | kanamycin-resistant gene | |
| Transformation efficiency | | |
| Kanamycin resistance basis | 4.0% | 2.5% |
| GUS activity basis | 3.3% | 1.9% |

### EXAMPLE 2

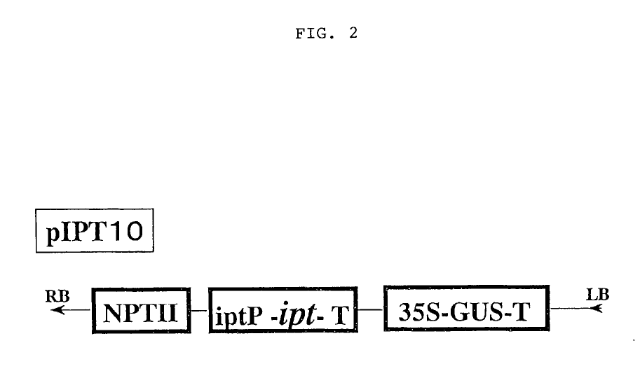

Instead of the kanamycin-resistant gene of pBI121 vector used in Example 1, a pIPT10 vector having A. tumefaciens PO22-derived ipt gene (its preparation method is described in JP-A-11-197722, cf. FIG. 2) was introduced as the selectable marker gene into the hypocotyl base of E. camaldulensis prepared by cutting off roots and seed leaves and keeping only the apical bud on its one end, and adventitious bud was differentiated in the same manner as in Example 1. In FIG. 2, iptP-ipt-T indicates the ipt gene to which the promoter of the ipt gene itself and a terminator are connected at the 5′ side and at the 3′ side, respectively, and the others are the same as in FIG. 1. In this case, however, the hypocotyl was precultured for 1 day using the shoot regeneration medium prior to infection with Agrobacterium. Also, plant hormone and kanamycin were not added to the medium for adventitious bud differentiation after the Agrobacterium infection. Apical buds of hypocotyls were cut off after 10 weeks from the Agrobacterium infection.

When tests were carried out using 40 hypocotyls, growth of some calli was observed prior to the differentiation of adventitious buds in this case. Accordingly, the GUS activity test was carried out on the adventitious buds differentiated from calli and also on the calli themselves, 3 months after the Agrobacterium infection. As a result, the GUS activity was found in calli derived from 20 hypocotyls (hereinafter referred to as “calli of 20 lines”), 6 lines among the calli of 20 lines differentiated adventitious buds, and adventitious buds of 4 lines among them showed the GUS activity. That is, on the GUS activity basis, the transformation efficiency into calli was 20/40×100=50.0%, and the transformation efficiency into adventitious buds was 4/40×100=10.0%. The results are shown in Table 2.

### COMPARATIVE EXAMPLE 2

Adventitious buds were differentiated by introducing the GUS gene and the ipt gene using the pIPT10 vector in the same manner as in Example 2, except that those in which roots, cotyledons and apical buds were cut off from germinated seedlings of E. camaldulensis seeds were used as the hypocotyls for gene introduction.

When tests were carried out using 98 hypocotyls, growth of some calli was observed prior to the differentiation of adventitious buds in this case, too. As a result of the GUS activity test 3 months after the Agrobacterium infection, the GUS activity was found in calli of 40 lines, and 5 lines among the calli of 40 lines differentiated adventitious buds. However, the GUS activity was not able to be found in any of the adventitious buds. That is, on the GUS activity basis, the transformation efficiency into calli was 40/98×100=40.8%, and the transformation efficiency into adventitious buds was 0/98×100=0.0%. The results are shown in Table 2.

### EXAMPLE 3

Seeds of Eucalyptus globulus (hereinafter referred to as “E. globulus”) were sterilized by soaking them in 70% ethanol for 1 minute and further soaking in 2% aqueous sodium hypochlorite solution for about 4 hours with stirring, washed thoroughly with sterile water and inoculated onto a germination medium, preserved for 2 days or more in a refrigerator of 4° C. for accelerating germination, and then cultured at 25° C. under whole light conditions of 40 μmol/m²/s in light intensity for the germination. In this case, the MS medium was supplemented with 0.5 mg/l zeatin, 20 g/l sucrose and 9 g/l agar and used as the germination medium.

One to two weeks after the inoculation of seeds onto the germination medium, roots and cotyledons were cut off from the thus germinated seedlings to obtain the hypocotyls each keeping apical bud alone on its one end, and the GUS gene was introduced into its base part having no growing point, together with the kanamycin-resistant gene as a selectable marker gene. That is, the hypocotyl was soaked in a pBI121 vector (available from CLONTECH, cf. FIG. 1)-introduced Agrobacterium cell suspension prepared in the same manner as in Example 1, and after discarding excess cell suspension, cocultured with the Agrobacterium at 25° C. for 3 days in the dark using a medium for adventitious bud differentiation supplemented with 40 mg/l acetosyringon, to thereby infect it with the pBI121 vector-introduced Agrobacterium. In this case, the concentration of the nitrogen source alone in the modified MS medium was changed to ½, and the resulting medium was supplemented with 1.0 mg/l zeatin, 0.05 mg/l NAA, 20 g/l sucrose and 9 g/l agar and used as the medium for adventitious bud differentiation.

The hypocotyl after coculturing was transplanted onto the medium for adventitious bud differentiation further supplemented with 500 mg/l ticarcillin and 100 mg/l kanamycin for the selection of pBI121 vector-introduced cells and cultured at 25° C. under whole light conditions of about 40 μmol/m²/s in light intensity by sub-culturing it using the same medium composition at an interval of 2 weeks, to thereby differentiate adventitious buds. In this case, the apical bud of hypocotyl was cut off after 1 month of the Agrobacterium infection.

When tests were carried out on 182 hypocotyls, growth of some calli was observed prior to the differentiation of adventitious buds in this case, too. Accordingly, the GUS activity test was carried out on the adventitious buds differentiated from calli and also on the calli themselves, 3 months after the Agrobacterium infection. As a result, the GUS activity was found in calli of 40 lines, 12 lines among the calli of 40 lines differentiated adventitious buds, and adventitious buds of 6 lines among them showed the GUS activity. That is, on the GUS activity basis, the transformation efficiency into calli was 40/182×100=22.0%, and the transformation efficiency into adventitious buds was 6/182×100=3.3%. The results are shown in Table 2.

### COMPARATIVE EXAMPLE 3

Introduction of the GUS gene and kanamycin-resistant gene and subsequent regeneration of plants were carried out in the same manner as in Example 3, except that those which were prepared by cutting off roots, seed leaves and apical buds from the germinated seedlings of E. globulus seeds were used as the hypocotyls for gene introduction.

When tests were carried out on 220 hypocotyls, growth of some calli was observed prior to the differentiation of adventitious buds in this case, too. As a result of the GUS activity test carried out 3 months after the Agrobacterium infection, the GUS activity was found in calli of 9 lines, 2 lines among the calli of 9 lines differentiated adventitious buds, and the adventitious bud of 1 line among them showed the GUS activity. That is, on the GUS activity basis, the transformation efficiency into calli was 9/220×100=4.1%, and the transformation efficiency into adventitious buds was 1/220×100=0.5%. The results are shown in Table 2.

### EXAMPLE 4

The pIPT10 vector (its preparation method is described in JP-A-11-197722, cf. FIG. 2) was introduced into the hypocotyl base of E. globulus prepared by cutting off roots and seed leaves and keeping only the apical bud on its one end, and adventitious bud was differentiated in the same manner as in Example 3. In this case, however, plant hormone and kanamycin were not added to the medium for adventitious bud differentiation.

As a result of tests on 120 hypocotyls, adventitious buds were differentiated from 22 hypocotyls until 4 months after their infection with Agrobacterium, and the GUS activity was found in the adventitious buds of 8 lines among them. That is, the transformation efficiency into adventitious buds in this case was 8/120×100=6.7% based on the GUS activity. The results are shown in Table 2.

### COMPARATIVE EXAMPLE 4

Adventitious buds were differentiated by introducing the GUS gene and the ipt gene using the pIPT10 vector in the same manner as in Example 4, except that those which were prepared by cutting off roots, seed leaves and apical buds from the germinated seedlings of E. globulus seeds were used as the hypocotyls for gene introduction.

As a result of tests on 180 hypocotyls, adventitious buds were differentiated from 18 hypocotyls until 4 months after the infection with Agrobacterium, and the GUS activity was found in the adventitious buds of 6 lines among them. That is, the transformation efficiency into adventitious buds in this case was 6/180×100=3.3% based on the GUS activity. The results are shown in Table 2.

### EXAMPLE 5

Each stem of a hybrid aspen (Populus sieboldii×Populus grandidentata) Y-63 (collected from the experimental forests belonging to Akita Jujo Chemicals Co., Ltd.) growing in vitro was cut out keeping a node, inoculated onto a germination medium and cultured at 25° C. under a whole light condition of about 40 μmol/m²/s in light intensity, and the normal bud grown from the node was allowed to elongate to a length of about 1 cm. In this case, the modified MS medium was supplemented with 0.5 mg/l zeatin, 20 g/l sucrose and 9 g/l agar and used as the germination medium.

Next, the normal bud was cut out from around its base with the stem to obtain a short shoot having an apical bud on its tip, and the GUS gene was introduced into the base part of the shoot together with the ipt gene as a selectable marker gene. That is, the cutting face of the base part of the shoot, which was formed when the normal bud was cut out from the stem, was soaked in a 2-fold diluted suspension of the pIPT10 vector (its preparation method is described in JP-A-11-197722, cf. FIG. 2)—introduced Agrobacterium prepared in the same manner as in Example 1, and after discarding excess cell suspension, the shoot was inoculated into a medium for adventitious bud differentiation supplemented with 40 mg/l acetosyringon and cocultured with the Agrobacterium at 25° C. for 2 days in the dark, to thereby infect it with the pIPT10 vector-introduced Agrobacterium. In this case, the modified MS medium further supplemented with 20 g/l sucrose and 9 g/l agar was used as the medium for adventitious bud differentiation.

The shoot after the coculturing was transplanted onto the medium for adventitious bud differentiation further supplemented with 500 mg/l carbenicillin and cultured at 25° C. under whole light conditions of about 40 μmol/m²/s in light intensity by subculturing it using the same medium composition at an interval of 10 days, to thereby differentiating adventitious buds. In this case, the apical bud of the shoot tip was cut off after 1 month of the Agrobacterium infection.

As a result of the test on 30 shoots, adventitious buds were differentiated from 25 shoots until 40 days after the infection with Agrobacterium, and the GUS activity was found in the adventitious buds of 8 lines among them. That is, the transformation efficiency into adventitious buds in this case was 8/30×100=26.7% based on the GUS activity. The results are shown in Table 2.

### COMPARATIVE EXAMPLE 5

Adventitious buds were differentiated by introducing the GUS gene and the ipt gene in the same manner as in Example 5, except that a shoot having a length of about 5 mm prepared by cutting off the apical bud of its tip part was used as the segment for gene introduction use.

As a result of the test on 43 segments, adventitious buds were differentiated from 25 segments until 40 days after their infection with Agrobacterium, and the GUS activity was found in the adventitious buds of 4 lines among them. That is, the transformation efficiency into adventitious buds in this case was 4/43×100=9.3% based on the GUS activity. The results are shown in Table 2.

TABLE 2

| | | Comp. | | Comp. |
|---|---|---|---|---|
| | Ex. 2 | Ex. 2 | Ex. 3 | Ex. 3 |
| Plant | E. camaldulensis | | E. globulus | |
| Desired gene | GUS gene | | GUS gene | |
| Selectable marker gene | ipt gene | | kanamycin-resistant | |
| | | | gene | |
| Transformation efficiency | | | | |
| (GUS activity basis) | | | | |
| Callus | 50.0% | 40.8% | 22.0% | 4.1% |
| Adventitious bud | 10.0% | 0.0% | 3.3% | 0.5% |
| | | Comp. | | Comp. Ex. |
| | Ex. 4 | Ex. 4 | Ex. 5 | 5 |
| Plant | E. camaldulensis | | hybrid aspen | |
| Desired gene | GUS gene | | GUS gene | |
| Selectable marker gene | ipt gene | | ipt gene | |
| Transformation efficiency | | | | |
| (GUS activity basis) | | | | |
| Callus | — | — | — | — |
| Adventitious bud | 6.7% | 3.3% | 26.7% | 9.3% |

As is apparent from Tables 1 and 2, all of the Examples 1 to 5 showed higher transformation efficiency on the kanamycin resistance basis and GUS gene expression basis than the respective corresponding Comparative Examples 1 to 5.

Also, the GUS gene-introduced calli in Examples 2 and 3 showed higher probability to differentiate GUS gene-introduced adventitious buds than those in Comparative Examples 2 and 3. Furthermore, the probability in Example 2 is 4/20×100=20.0% because GUS gene-introduced adventitious buds were differentiated from 4 lines among 20 lines of the GUS gene-introduced calli, the probability in Comparative Example 2 is 0/40×100=0% because 40 lines of GUS gene-introduced calli were obtained but all of them did not differentiate GUS gene-introduced adventitious bud, the probability in Example 3 is 6/40×100=15.0% because GUS gene-introduced adventitious buds were differentiated from 6 lines among 40 lines of the GUS gene-introduced calli, and the probability in Comparative Example 3 is 1/9×100=11.1% because a GUS gene-introduced adventitious bud was differentiated from 1 line among 9 lines of the GUS gene-introduced calli.

As the reason for this, it is considered that the GUS gene-introduced calli in Examples 2 and 3 actively differentiated adventitious buds as a whole in comparison with the GUS gene-introduced calli in Comparative Examples 2 and 3. The adventitious bud differentiation ratio from the GUS gene-introduced calli supports the reason. That is, the adventitious bud differentiation ratio in these cases was 6/20×100=30% in Example 2, 5/40×100=12.5% in Comparative Example 2, 12/40×100=30% in Example 3 and 2/9×100=22.2% in Comparative Example 3. It is considered that such an improvement in the adventitious bud differentiation ratio is due to an influence of the apical bud kept on one end of the hypocotyl used as a tissue for gene introduction in Examples 2 and 3.

While the invention has been described in detail and with reference to specific examples thereof, it will be apparent to one skilled in the art that various changes and modifications can be made therein without departing from the spirit and scope thereof. All references cited herein are incorporated in their entirety.

The priority application, Japanese patent application No. 2001-223664, filed Jul. 24, 2001, is incorporated herein by reference in their entirety.

What is claimed is:

1. A method for producing a transgenic plant, which comprises:
preparing a shoot having at least one end having a growing point and a base part having no growing point as a tissue for gene introduction; and
introducing a desired gene into the base part while keeping the growing point in the at least one end to differentiate an adventitious bud.

2. The method according to claim 1, wherein the shoot is a shoot of the genus Eucalyptus.

3. The method according to claim 1, wherein the shoot is a hypocotyl having an apical bud or an apical bud primordium.

4. The method according to claim 1, wherein the desired gene is introduced into the end face of the base part of the shoot generated when a shoot is prepared as the tissue for gene introduction.

5. The method according to claim 1, wherein the desired gene is introduced using an Agrobacterium method.

6. The method according to claim 1, wherein a cytokinin-related gene is introduced as a selectable marker gene together with the desired gene.

7. The method according to claim 6, wherein the cytokinin-related gene is the ipt (isopentenyl transferase) gene.
